(19) United States
(12) Patent Application Publication
(10) Pub. No.: US 2006/0078931 A1
(43) Pub. Date: Apr. 13, 2006

(54) Microchip unit, and method of conducting biochemical reaction using the microchip unit

(76) Inventors: Kwang-wook Oh, Gyeonggi-do (KR); Yu-jin Seo, Gyeonggi-do (KR); Gyeong-sik Ok, Busan (KR); Jin-tae Kim, Gyeonggi-do (KR); Kak Namkoong, Seoul (KR); Chin-sung Park, Gyeonggi-do (KR)

Correspondence Address: CANTOR COLBURN, LLP, 55 GRIFFIN ROAD SOUTH, BLOOMFIELD, CT, 06002, US

(21) Appl. No.: 11/245,348

(22) Filed: Oct. 6, 2005

(30) Foreign Application Priority Data
Oct. 7, 2004 (KR) 10-2004-0079957

(51) Int. Cl. C12Q 1/68; C12M 1/34

(52) U.S. Cl. 435/6; 435/287.2

(57) ABSTRACT

Provided is a microchip unit, including a microchip on which a plurality of micro-channels are formed, a housing disposed below the microchip to fix the microchip; and at least two injecting and sealing elements having through-holes corresponding to inlets of the microchip. The injecting and sealing elements are vertically fixed on the top of the housing and slide in a horizontal direction from a first location to a second location and vice versa. The through-holes are aligned with inlets of the microchip so that a reaction solution can be injected through the through-holes when the injecting and sealing elements are placed at the first location. The inlets of the microchip are sealed by elastic members formed on bottom surfaces of the injecting and sealing elements when the injecting and sealing elements are placed at the second location.

### BACKGROUND OF THE INVENTION

This application claims the priority of Korean Patent Application No. 10-2004-0079957, filed on Oct. 7, 2004 in the Korean Intellectual Property Office, the disclosure of which is incorporated herein in its entirety by reference.

1. Field of the Invention

The present invention relates to a microchip unit, and a method of conducting a biochemical reaction using the microchip unit.

2. Description of the Related Art

Conventional micro-channels and microchips including chambers in which a biochemical reaction can occur are well known. An example of a microchip is a polymerase chain reaction (PCR) chip in which a micro-channel and a reaction chamber are formed. In conventional microchips, injection equipment such as a pipette is used to directly inject reaction solutions directly into inlets of the microchips. That is, a pipette is used to manually inject a PCR solution into an inlet or an outlet of a conventional PCR chip. However, when a multi-channel PCR chip having a plurality of reaction chambers is used, such a manual operation can result in the PCR solution being injected into wrong PCR channels of the multi-channel PCR chip. In addition, microchips must be sealed after a PCR solution is injected so that the PCR solution is not lost by, for example, evaporation while a PCR is performed. Thus, to prevent the loss of the PCR solution, tape is adhered to a surface on which inlets and/or outlets of the PCR chip are formed, or a sealing material is used to seal the surface. Therefore, according to the conventional art, a process of manually injecting the PCR solution and a process of sealing the inlets and/or outlets of the PCR chip using, for example, tape after injecting the PCR solution must be included.

Although the conventional method can be used for a single channel PCR chip, it is inconvenient to use for a multi-channel PCR chip. Therefore, a method and apparatus for easily and accurately injecting a PCR solution and simply sealing an inlet and/or outlet of a multi-channel PCR chip after injecting the PCR solution are required.

Therefore, a semiautomatic operating device for a microchip in which a reaction solution can be simply and accurately injected and a solution inlet and outlet can be easily sealed after injecting the reaction solution through a simple manipulation of the device regardless of the level of the skill of a user is required.

The inventors of the present application have completed the present invention while researching methods of simply and accurately injecting a PCR solution into a multi-channel PCR chip.

### SUMMARY OF THE INVENTION

The present invention provides a microchip unit that can simply and accurately inject a reaction solution into micro-channels of a microchip unit.

The present invention also provides a method of conducting a biochemical reaction using the microchip unit.

According to an aspect of the present invention, there is a microchip unit, comprising:
a microchip in which a plurality of micro-channels are formed;
a housing disposed below the microchip to fix the microchip; and
at least two injecting and sealing elements that have through-holes corresponding to inlets of the microchip,
wherein the injecting and sealing elements vertically fixed on top of the housing, slide in a horizontal direction from a first location to a second location and vice versa, the through-holes being aligned with inlets of the microchip so that a reaction solution can be injected through the through-holes when the injecting and sealing elements are placed at the first location, and the inlets of the microchip being sealed by elastic members formed on bottom surfaces of the injecting and sealing elements when the injecting and sealing elements are placed at the second location.

According to another aspect of the present invention, there is provided a method of performing a biochemical reaction using a microchip unit including a microchip in which a plurality of micro-channels are formed; a housing disposed below the microchip to fix the microchip; and at least two injecting and sealing elements that have through-holes corresponding to inlets of the microchip, wherein the injecting and sealing elements vertically fixed on top of the housing, slide in a horizontal direction from a first location to a second location and vice versa, the through-holes being aligned with inlets of the microchip so that a reaction solution can be injected through the through-holes when the injecting and sealing elements are placed at the first location, and the inlets of the microchip being sealed by elastic members formed on bottom surfaces of the injecting and sealing elements when the injecting and sealing elements are placed at the second location, the method comprising:
sliding the injecting and sealing elements to the first location to inject the reaction solution via the through-holes; and
sliding the injecting and sealing elements to the second location to seal the inlets of the microchip.

### BRIEF DESCRIPTION OF THE DRAWINGS

The above and other features and advantages of the present invention will become more apparent by describing in detail exemplary embodiments thereof with reference to the attached drawings in which:

FIG. 1 is a perspective view of a polymerase chain reaction (PCR) chip unit including two injecting and sealing elements disposed in a first location according to an embodiment of the present invention;

FIG. 2 is a perspective view of the PCR chip unit of FIG. 1 when the injecting and sealing elements are disposed in a second location;

FIG. 3 is an exploded perspective view of the PCR chip unit according to one embodiment of the present invention as shown in FIG. 1;

FIG. 4 is a cross-section of the injecting and sealing element taken along line 2-2′ in FIG. 3;

FIG. 5 is a cross-section of the PCR chip unit taken along line 4-4′ in FIG. 1 when a PCR solution is injected into the PCR chip unit using a pipette and the injecting and sealing elements are disposed in the first location, that is, an injection mode; and

FIG. 6 is a cross-section of the PCR chip unit taken along line 6-6′ in FIG. 2 when the injecting and sealing elements are disposed in the second location, that is, a sealing mode.

### DETAILED DESCRIPTION OF THE INVENTION

According to an aspect of the present invention, there is provided a microchip unit including a microchip on which a plurality of micro-channels are formed, a housing disposed below the microchip to fix the microchip, and at least two injecting and sealing elements including through-holes corresponding to inlets of the microchip. The injecting and sealing elements are vertically fixed on top of the housing and slide in a horizontal direction from a first location to a second location and vice versa. The through-holes are aligned with inlets of the microchip so that a reaction solution can be injected through the through-holes when the injecting and sealing elements are placed at the first location. The inlets of the microchip are sealed by elastic members formed on bottom surfaces of the injecting and sealing elements when the injecting and sealing elements are placed at the second location.

The microchip unit of the present invention may be a PCR chip unit including a PCR chip on which a plurality PCR channels are formed, a housing disposed below the PCR chip to fix the PCR chip; and at least two injecting and sealing elements having through-holes corresponding to inlets of the PCR chip. The injecting and sealing elements are vertically fixed on top of the housing and slide in a horizontal direction from a first location to a second location and vice versa. The through-holes are aligned with inlets of the PCR chip so that a reaction solution can be injected through the through-holes when the injecting and sealing elements are placed at the first location. The inlets of the PCR chip are sealed by elastic members formed on bottom surfaces of the injecting and sealing elements when the injecting and sealing elements are placed at the second location.

According to an aspect of the present invention, there is provided a method of performing a biochemical reaction using a microchip unit including a microchip on which a plurality of micro-channels are formed; a housing disposed below the microchip, fixing the microchip; and at least two injecting and sealing elements having through-holes corresponding to inlets of the microchip, wherein the injecting and sealing elements vertically fixed on the top of the housing, slides in a horizontal direction from a first location to a second location and vice versa, in which the through-holes are aligned with inlets of the microchip so that a reaction solution can be injected through the through-holes when the injecting and sealing elements are placed at the first location, and the inlets of the microchip are sealed by elastic members formed on bottom surfaces of the injecting and sealing elements when the injecting and sealing elements are placed at the second location. The method includes: sliding the injecting and sealing elements to the first location to inject the reaction solution via the through-holes; and sliding the injecting and sealing elements to the second location to seal the inlets of the microchip.

An example of the method of performing the biochemical reaction using the microchip unit in the present invention includes a PCR chip unit having a PCR chip on which a plurality of PCR channels are formed; a housing disposed below the PCR chip, fixing the PCR chip; and at least two injecting and sealing elements having through-holes corresponding to inlets of the PCR chip, wherein the injecting and sealing elements vertically fixed on the top of the housing, slides in a horizontal direction from a first location to a second location and vice versa, in which the through-holes are aligned with inlets of the PCR chip so that a reaction solution can be injected through the through-holes when the injecting and sealing elements are placed at the first location, and the inlets of the PCR chip are sealed by elastic members formed on bottom surfaces of the injecting and sealing elements when the injecting and sealing elements are placed at the second location. The method includes: sliding the injecting and sealing elements to the first location to inject the reaction solution via the through-holes; and sliding the injecting and sealing elements to the second location to seal the inlets of the PCR chip.

The method further includes conducting thermal cycling reaction after fixing the sealed microchip unit in a thermal cycler as a module.

The term “microchip” used throughout the specification denotes a device including a micro-channel and a chamber that is in fluid communication with the micro-channel and can be opened or closed from the micro-channel so that various biochemical reactions can be performed in the chamber using a small amount of a reaction solution. Such a microchip is well known to those skilled in the prior art related to the present invention. An example of the microchip is a PCR chip in which a micro-channel and a reaction chamber that can be in fluid communication with the micro-channel are formed.

The PCR chip used in the present invention is well known to those skilled in the prior art related to the present invention. Generally, a “PCR chip” refers to a device including a micro-channel and a micro chamber in which a micro PCR can be performed. The PCR chip may be a single PCR chip having a single channel and chamber, or a multi-channel PCR chip having a plurality of channels and chambers.

Throughout the specification, a “PCR,” an acronym for polymerase chain reaction, is a process in which a target nucleotide is amplified from a pair of primers specifically bound to the target nucleotide using the polymerase. In a PCR, a polymerase, a primer, a template, and a solution including other subsidiary elements (a.k.a. “PCR mixture”) are injected into a chamber. Then, the contents of the chamber are maintained at an annealing temperature at which the primer and the template are annealed, then at a polymerizating temperature at which polymerization occurs by the polymerase, and then at a denaturizing temperature at which the polymerized double strands are denatured into single strands, for predetermined periods of time. A target nucleotide is amplified by repeating the temperature cycle mentioned above. A PCR is also known as a thermal cycling reaction. The PCR chip used in the present invention may be a well-known PCR chip.

In the present invention, the microchip fixing elements are formed on a housing and includes fixing elements which vertically fix the injecting and sealing elements and enables sliding of the injecting and sealing elements in the horizontal direction. The housing and the microchip or the housing and the injecting and sealing elements may be fixed by any fixing elements. They may also be fixed by meshing elements.

In the present invention, the injecting and sealing elements include through-holes corresponding to the inlets of the microchip. Although the injecting and sealing elements cannot slide up or down since they are fixed to the housing, the injecting and sealing elements can slide from a first location to a second location. When the injecting and sealing elements are at the first location, the through-holes are aligned with the inlets of the microchip, and thus a reaction solution can be injected via the through-holes. When the injecting and sealing elements are at the second location, each of the inlets of the microchip is sealed by elastic members formed on bottom surfaces of the injecting and sealing elements. In the present invention, the elastic members may be composed of any material with elasticity, for example, rubber or PDMS. Preferably, the elastic members are PDMS.

In a method of conducting a biochemical reaction using the microchip unit, a reaction solution is injected into micro-channels and/or chambers of a microchip via through-holes and inlets of the microchip unit using injection equipment such as a pipette after sliding the injecting and sealing elements of the microchip unit to a first location so that the through-holes and the inlets are aligned with each other. Next, the injecting and sealing elements are slide to a second location so that the elastic members formed on the bottom surfaces of the injecting and sealing elements contact the inlets of the microchip. As a result, the inlets are sealed. When the inlets of the microchip are sealed by injecting and sealing elements of the present invention when the reaction solution is in the chambers, the microchip unit can be connected to a conventional thermal cycler, for example, to perform PCR. For example, a PCR chip in which a PCR solution is injected in chambers and inlets are sealed can be fixed in a particular thermal cycler as a single module so that a thermal cycling reaction can occur.

The present invention will now be described more fully with reference to the accompanying drawings, in which exemplary embodiments of the invention are shown. Like reference numerals in the drawings denote like elements.

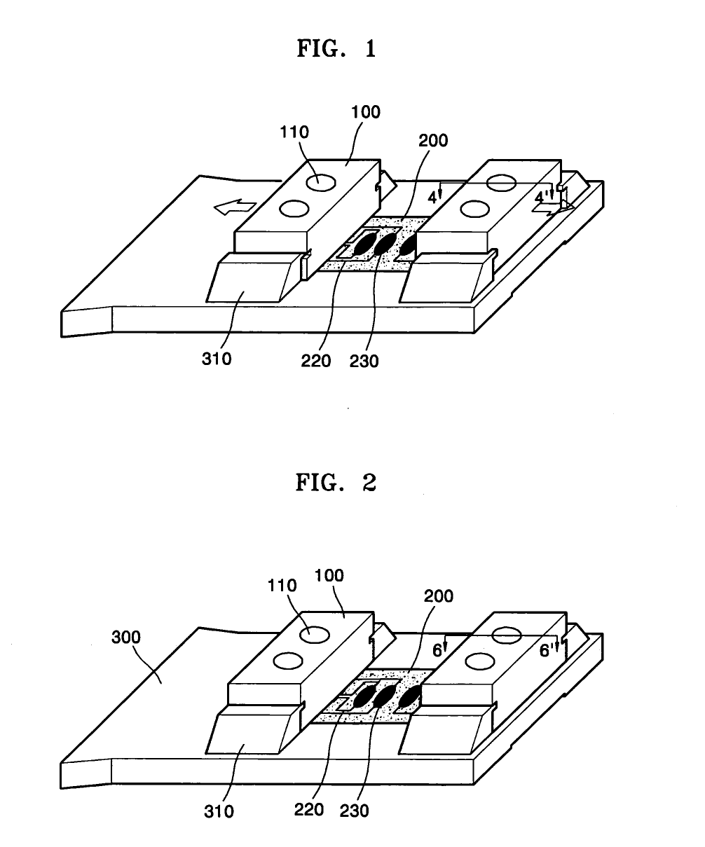

FIG. 1 is a perspective view of a polymerase chain reaction (PCR) chip unit including two injecting and sealing elements 100 disposed in a first location according to an embodiment of the present invention. Referring to FIG. 1, micro-channels 220 and micro chambers 230 are formed in a PCR chip 200, and thus PCR can be performed using an element providing a thermal cycling. The PCR chip 200 is fixed on a housing 300 including the injecting and sealing elements 100 and fixing elements 310. The injecting and sealing elements 100, in which through-holes 110 are formed, are vertically fixed on the top of the PCR chip 200 and the housing 300 via the fixing elements 310. The through-holes 110 are aligned with inlets of the PCR chip 200 when the injecting and sealing elements 100 are at the first location. Thus, the PCR solution can be injected into the micro-channels 220 and/or the chambers 230 of the PCR chip 200 through the through-holes 110 using injection equipment such as a pipette and propagate. In the present embodiment, the injecting and sealing elements 100 are vertically fixed on top of the housing 300 via the fixing elements 310. However, the fixing elements 310 can be any other elements which vertically fix the injecting and sealing elements 100 while enabling sliding of the injecting and sealing elements 100 in the horizontal direction.

FIG. 2 is a perspective view of the PCR chip unit of FIG. 1 when the two injecting and sealing elements 100 are disposed in a second location. When the injecting and sealing elements 100 are located at the first location, as in FIG. 1, and slide in directions indicated by arrows illustrated in FIG. 1 by applying a force to the injecting and sealing elements 100, the injecting and sealing elements 100 move to the second location illustrated in FIG. 2. By sliding the injecting and sealing elements 100 from the first location to the second location, elastic members 120 (see FIG. 4) formed on the bottom surfaces of the injecting and sealing elements 100 seal the inlets 210 of the PCR chip 200. Pressure is vertically applied to the inlets 210 sealed in this way. That is, the inlets 210 are sealed by the elastic members 120 with sufficient pressure to ensure that PCR solution does not leak during a PCR reaction.

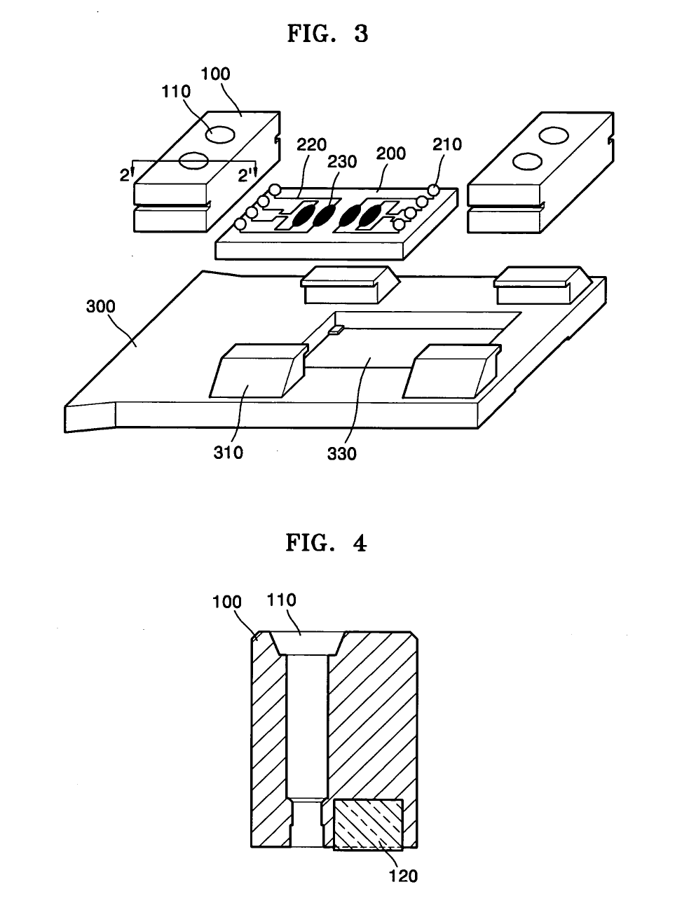

FIG. 3 is an exploded perspective view of the PCR chip unit illustrated in FIGS. 1 and 2. Referring to FIG. 3, the PCR chip unit comprises the two injecting and sealing elements 100, the PCR chip 200, and the housing 300. The PCR chip 200 is fixed to a PCR chip fixing unit 330 of the housing 300 on which the fixing elements 310 are formed. The PCR chip 200 comprises inlets and/or outlets 210 through which the PCR solution and/or reaction product is injected or output, the micro-channels 220, and the chambers 230, and these components are connected to one another. After the PCR chip 200 is fixed to the housing 300, the injecting and sealing elements 100 are fixed vertically on top of the fixing elements 310 and slide in the horizontal direction from the first location to the second location and vice versa.

FIG. 4 is a cross-section of the injecting and sealing element 100 taken along line 2-2′ in FIG. 3. Referring to FIG. 4, the through-holes 110 are formed in the injecting and sealing elements 100, and bottoms of the through-holes 110 are aligned with the inlets 210 of the PCR chip 200 when the injecting and sealing elements 100 are in the first location, thereby allowing the PCR solution to freely flow into the inlets 210. Therefore, when the injecting and sealing elements 100 are disposed in the first location, the PCR solution can be injected into the micro-channels 220 and the chambers 230 of the PCR chip 200 by injecting the PCR solution into the through-holes 110 using an injection device such as a pipette. The elastic members 120 may be formed of PDMS or rubber on the bottom surfaces of the injecting and sealing elements 100. The elastic members 120 may protrude from the bottom surfaces of the injecting and sealing elements 100 so that a predetermined pressure can be applied to the PCR chip 200 in a downward direction.

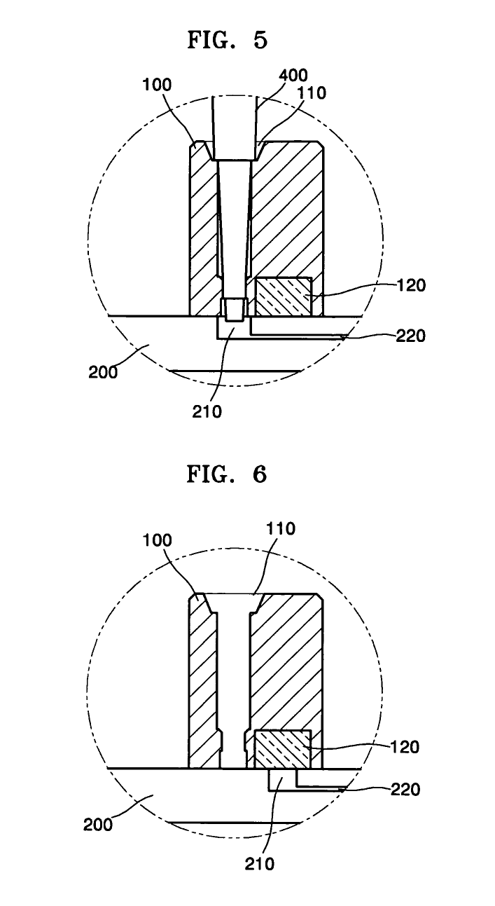

FIG. 5 is a cross-section of the PCR chip unit taken along line 4-4′ in FIG. 1 when the PCR solution is injected into the PCR chip unit using a pipette 400 while the injecting and sealing elements 100 are disposed in the first location, that is, when the injecting and sealing elements 100 are in an injection mode. As illustrated in FIG. 5, the PCR solution is injected from the pipette 400 into one of the inlet 210s of the PCR chip 200 through the corresponding through-hole 110. The injected PCR solution travels into the chamber 230 via the micro-channel 220. At this time, the elastic members 120 on the bottom surfaces of the injecting and sealing elements 100 are not in contact with the inlets 210.

FIG. 6 is a cross-section of the PCR chip unit taken along line 6-6′ in FIG. 2 when the injecting and sealing elements 100 are disposed in the second location. As illustrated in FIG. 6, by sliding the injecting and sealing elements 100 in the horizontal direction after the PCR solution is injected, the elastic members 120 on the bottom surfaces of the injecting and sealing elements 100 come in contact with the inlets 210 of the PCR chip 200, thereby sealing the inlets 210. The elastic members 120 apply a predetermined pressure in the downward direction such that the elastic members 120 are coupled to the PCR chip unit, thereby preventing leakage of the PCR solution from the inlets 210 during PCR. The elastic members 120 apply a predetermined pressure in the downward direction because the elastic members 120 protrude from the bottom surfaces of the injecting and sealing elements 100, which can be explicitly seen when the injecting and sealing elements 100 are not coupled to the PCR chip unit. The PCR solution does not leak from the inlets 210 during PCR due to the predetermined pressure.

According to a microchip unit of the present invention, a reaction solution can be injected into a micro-channel without being injected into incorrect micro-channels, and the microchip unit can be fixed and sealed using a simple method. Therefore, commonly used conventional processes of adhering tape to or sealing each of the inlets or outlets of a microchip unit after injecting a reaction solution are not used.

According to a method of conducting a biochemical reaction using the microchip unit, the reaction solution can be easily injected into a microchip and the microchip can be easily sealed. Thus, the biochemical reaction can be performed faster and easier.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A microchip unit, comprising:
a microchip in which a plurality of micro-channels are formed;
a housing disposed below the microchip to fix the microchip; and
at least two injecting and sealing elements that have through-holes corresponding to inlets of the microchip,
wherein the injecting and sealing elements vertically fixed on top of the housing, slide in a horizontal direction from a first location to a second location and vice versa, the through-holes being aligned with inlets of the microchip so that a reaction solution can be injected through the through-holes when the injecting and sealing elements are placed at the first location, and the inlets of the microchip being sealed by elastic members formed on bottom surfaces of the injecting and sealing elements when the injecting and sealing elements are placed at the second location.

2. The microchip unit of claim 1, wherein the microchip is a PCR chip.

3. The microchip unit of claim 1, wherein the elastic members are composed of PDMS.

4. A method of performing a biochemical reaction using a microchip unit including a microchip in which a plurality of micro-channels are formed; a housing disposed below the microchip to fix the microchip; and at least two injecting and sealing elements that have through-holes corresponding to inlets of the microchip, wherein the injecting and sealing elements vertically fixed on top of the housing, slide in a horizontal direction from a first location to a second location and vice versa, the through-holes being aligned with inlets of the microchip so that a reaction solution can be injected through the through-holes when the injecting and sealing elements are placed at the first location, and the inlets of the microchip being sealed by elastic members formed on bottom surfaces of the injecting and sealing elements when the injecting and sealing elements are placed at the second location, the method comprising:
sliding the injecting and sealing elements to the first location to inject the reaction solution via the through-holes; and
sliding the injecting and sealing elements to the second location to seal the inlets of the microchip.

5. The method of claim 4, further comprising performing thermal cycling reaction after fixing the sealed microchip unit in a thermal cycler as a module.

6. The method of claim 4, wherein the microchip is a PCR chip, and the biochemical reaction is a PCR.

7. The method of claim 4, wherein the elastic members are composed of PDMS.
